(19) United States
(12) Patent Application Publication
(10) Pub. No.: US 2018/0344892 A1
(43) Pub. Date: Dec. 6, 2018

(54) METHODS OF ACL REPAIR USING BIOLOGICALLY ACTIVE SUTURE

(71) Applicant: Smith & Nephew, Inc., Memphis, TN (US)

(72) Inventors: Nicholas John Cotton, Memphis, TN (US); Chen-rei Wan, Memphis, TN (US); John Stroncek, Memphis, TN (US)

(73) Assignee: Smith & Nephew, Inc., Memphis, TN (US)

(21) Appl. No.: 15/778,148

(22) PCT Filed: Nov. 18, 2016
(86) PCT No.: PCT/US16/62798

§ 371 Date: May 22, 2018

Related U.S. Application Data
(60) Provisional application No. 62/260,879, filed on Nov. 30, 2015.

(51) Int. Cl. A61L 17/00; A61B 17/04; A61B 17/06; A61B 17/16; A61F 2/08; A61L 17/04

(52) U.S. Cl. CPC A61L 17/005; A61B 17/0401; A61B 17/06166; A61B 2017/00893; A61F 2/0811; A61L 17/04; A61B 17/1675

(57) ABSTRACT

A method of repairing a partial or complete ACL tear using a biologically active suture combined with a suture anchor. The biologically active material may be an angiogenic material that provides a biological stimulus (such as blood vessel growth) to initiate the repair cascade throughout the tear.

### CROSS-REFERENCE TO RELATED APPLICATIONS

This application claims the benefit of U.S. Provisional Application Ser. No. 62/260879 filed 30 Nov. 2015 titled “Methods of ACL Repair Using Biologically Active Suture.” and PCT Application PCT/US16/062798, filed 18 Nov. 2016. The provisional and PCT are incorporated by reference herein as if reproduced in full below.

### TECHNICAL FIELD

This present disclosure relates to a method of tissue repair, in particular to a method of anterior cruciate ligament (ACL) repair and reconstruction using a biologically active suture.

### BACKGROUND

Arthroscopic surgery is a minimally-invasive surgery that involves the repair of tissue inside or around a joint. In the knee, for example, a common injury is a tear in the anterior cruciate ligament (ACL) extending between the femur and the tibia, which may be a partial or a complete thickness tear. Currently, there are no universally accepted therapies for partial ACL tears that do not respond to conservative treatment. Unfortunately, untreated partial tears can progress to complete thickness tears, which require ACL reconstruction surgery to rebuild the ligament in the knee. Current methods of ACL reconstruction often require the stabilization of the entire knee and/or the use of a graft, which adds additional time during the surgical procedure and subsequent recovery, as well as problems associated with graft morbidity.

One aim of medical practitioners following ACL repair is to incite rapid healing and tissue repair throughout the treatment site. A factor in the promotion of tissue repair is the extent to which reparative cells and other factors can permeate through to the tissue in question. This, in turn, is dependent upon the extent to which blood vessels can form in and around the site. The growth of new blood vessels from existing ones is known as “angiogenesis.”

### SUMMARY

Described herein are methods of repairing a partial or complete ACL tear using a biologically active suture combined with a knotted or knotless suture anchor. The biologically active suture provides a biological stimulus (such as angiogenesis) to initiate the repair cascade throughout the tear in moderately avascular tissue. Advantageously, some of the methods described herein provide mechanical stability to the repair site rather than to the entire knee, and may be used with or without a graft. Additionally, the methods described herein are quicker than conventional ACL reconstruction and may reduce rehabilitation time for the patient. Some methods described herein may reduce rehabilitation time for the patient in an ACL reconstruction involving a tissue graft.

In one example, a method of repairing an ACL tear may include, using a surgical technique, placing a first fixation device in one of a tibia or a femur near a first ACL insertion site. The first fixation device is attached to a second fixation device by a suture material. The method may also include, using the surgical technique, placing the second fixation device in the other of the tibia or the femur near a second ACL insertion site, such that the suture material is passed through the ACL and woven within a tear. The suture material may be comprised of a water soluble or water miscible, biologically active material or precursor thereof in admixture with a non-absorbable hydrophobic polymer which may stimulate tissue repair in the surrounding tissue.

In further examples, the biologically active material may be at least one of angiogenic material or angiogenic precursor material which is capable of breaking down in vivo to form angiogenic material, wherein the angiogenic material is in admixture with polypropylene. The angiogenic material may be one or more of butyric acid, butyric acid salt, α-monobutyrin; α-dibutyrin, β-dibutyrin, tributyrin, or hydroxybutyrate. The butyric acid salt may be selected from sodium, potassium, calcium, ammonium, and lithium salts. In other examples, the angiogenic material may be one or more of the following angiogenic factors: angiogenic peptide growth factors, including autologous, xenogenic, recombinant, and synthetic forms of these, including the vascular endothelial growth factors VEGF 121, 165, 189 and 206; fibroblast growth factors FGF-1, FGF-2, FGF-7 (keratinocyte growth factor); transforming growth factor family (TGF-α, -β, platelet derived growth factors PDGF-AA, PDGF-BB, and PDGF-AB; platelet derived endothelial cell growth factor (PD-ECGF); hypoxia inducible factor-1 (HIF-1); scatter factor (SF, also known as hepatocyte growth factor or HGF); placenta growth factor (PIGF)-1, -2; tumor necrosis factor a (TNF-α); midkine; pleiotrophin; insulin-like growth factor-1; epidermal growth factor (EGF); endothelial cell growth factor (ECGF); endothelial stimulating angiogenic factor (ESAF); connective tissue growth factor (CTGF); CYR61; Angiogenin; or Angiotrophin. The angiogenic material may be one or more blood clot breakdown products, including thrombin, heparin, and autologous, allogeneic, xenogeneic, recombinant, and synthetic forms of these materials. The angiogenic material may be one or more of hyaluronan, para-thyroid hormone, angiopoietin 1, del-1, erythropoietin, fas (CD95), follistatin, macrophage migration inhibitory factor, monocyte chemoattractant protein-1, and nicotinamide. The angiogenic precursor material may be one or more of fibrin, including autologous, allogeneic, xenogeneic, recombinant and synthetic forms thereof, and hyaluronic acid.

In still further examples, the suture may be coated on at least one external surface with the biologically active material or the suture has the biologically active material impregnated into at least one region of the suture. Either one of the first and second fixation devices may be a suture, a surgical arrow, a staple, a dart, a bolt, a screw, a button, an anchor, a nail or a rivet, or a barbed surgical device. The surgical technique may be one of an “all-inside” technique, a trans-tibial technique, a medial port technique or an “outside-in” technique. In some examples, a graft may be attached to the suture material.

In a further example, a method of anterior cruciate ligament (ACL) reconstruction is disclosed and may include passing a suture material through and along a length of a tissue graft, the suture material comprising a water soluble or water miscible, biologically active material or precursor thereof in admixture with a non-absorbable hydrophobic polymer. Passing may be achieved with a needle and/or utilizing a suture passing device. The tissue graft and suture material may then be placed within a first prepared tunnel within the tibia and a second prepared tunnel within the femur. The tissue graft may be coupled or fixed in place within the first and second tunnel using a first and second fixation device respectively. At least one of these fixation devices may include a loop of flexible material that is coupled to both a portion of the tissue graft and a button-type device that is configured to lie on an outer cortex of the tibia and/or femur. At least one of the fixation devices may be a screw-type anchor that is inserted into one of the tunnels and directly couples a portion of the tissue graft with the bone tunnel. The biologically active material may stimulate tissue graft remodeling.

In a further example, a method of anterior cruciate ligament (ACL) reconstruction is disclosed, including the steps of drilling a first tunnel within one of a tibia or a femur near a first end of an ACL insertion site, and also drilling a second tunnel within the other of the tibia or the femur near a second end of the ACL insertion site. The first and second tunnel may be coaxial with each other. A suture material is passed through and along a length of a tissue graft and then both the suture material and tissue graft placed within and along both tunnels. The suture material is comprised of a water soluble or water miscible, biologically active material or precursor thereof in admixture with a non-absorbable hydrophobic polymer; the biologically active material stimulating tissue repair and remodeling of the tissue graft.

In a further example, a kit for improved ligament reconstruction is disclosed, including a drill for preparing a first tunnel within a femur, a first fixation device and a suture material. The suture material is configured to be passed through and along a length of a tissue graft and the first fixation is configured to secure the tissue graft and thereby the suture material within and along the first tunnel. The suture material includes an angiogenic material that stimulates tissue graft repair and remodeling.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The disclosure will be more fully understood by reference to the detailed description, in conjunction with the following figures, wherein:

FIG. 1 is an illustration of an example of the methods of this disclosure;

FIGS. 2-5 are examples of surgical techniques using the methods of this disclosure; and

FIG. 6 is an illustration of alternative examples of the methods of this disclosure using a tissue graft.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

Comprise, include, and/or plural forms of each are open ended and include the listed parts and can include additional parts that are not listed. And/or is open ended and includes one or more of the listed parts and combinations of the listed parts.

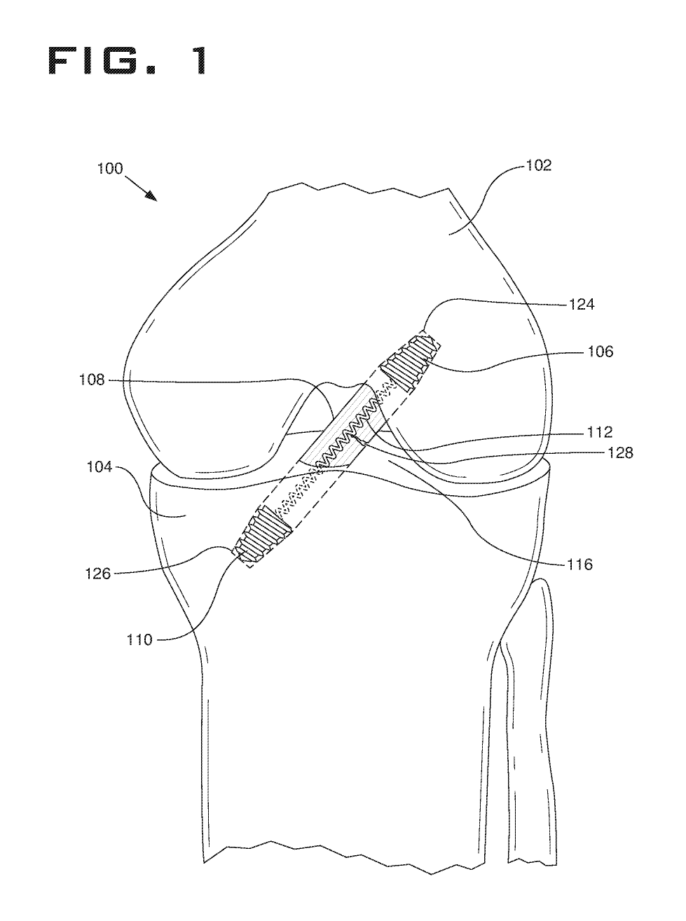

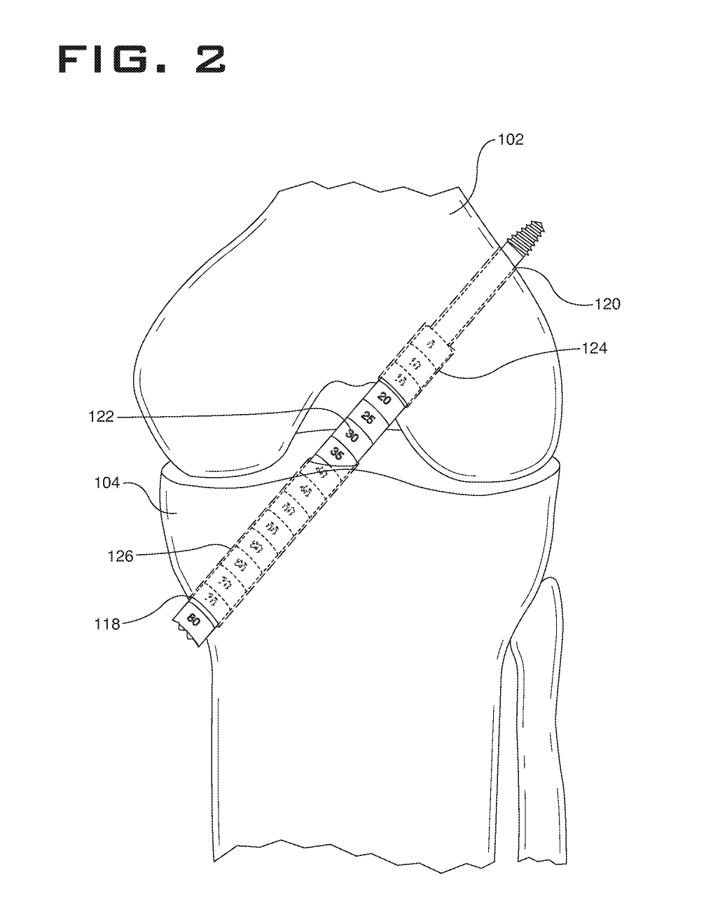

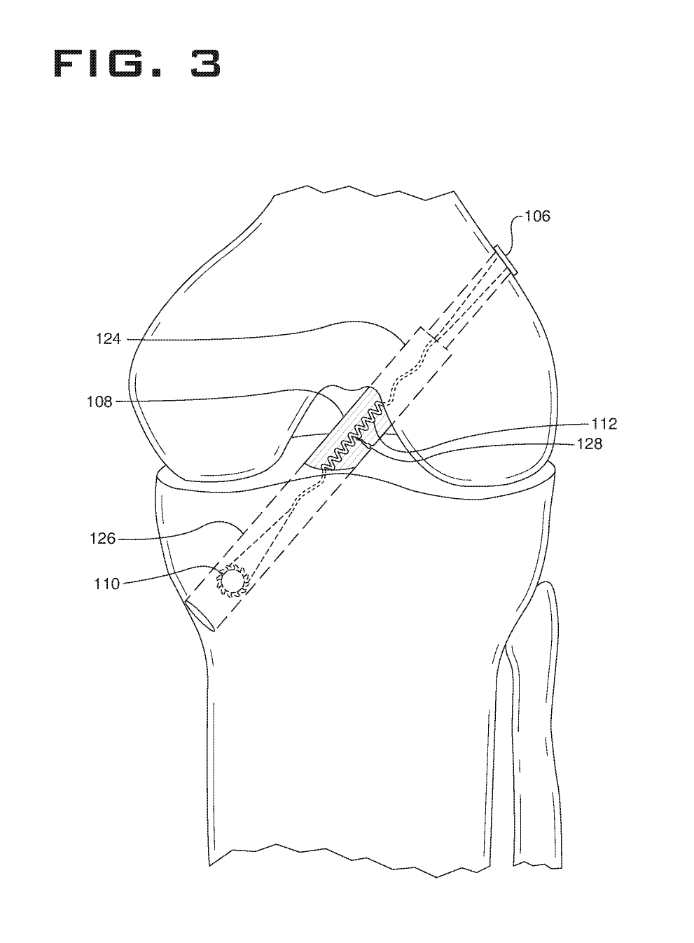

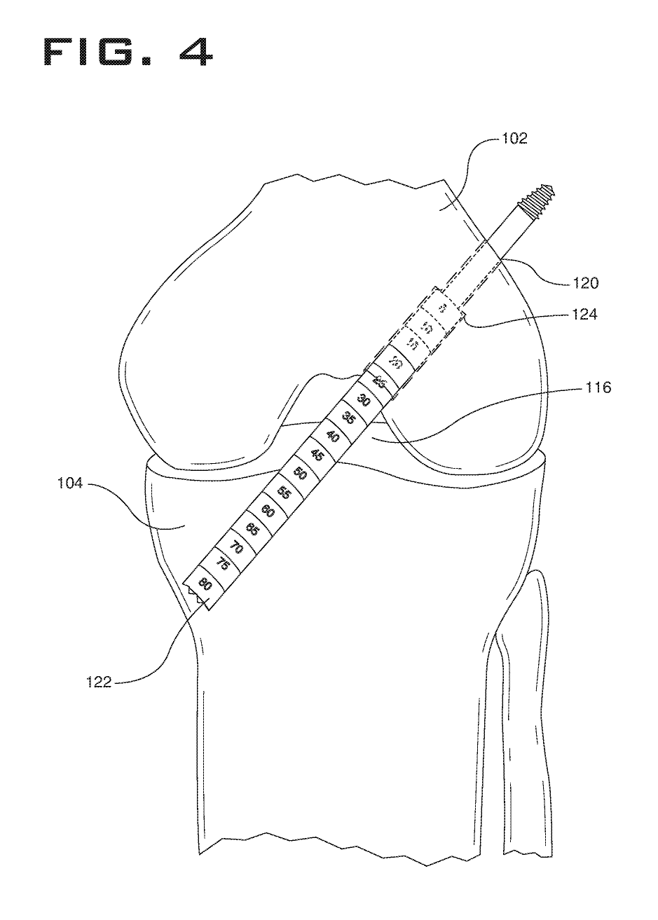

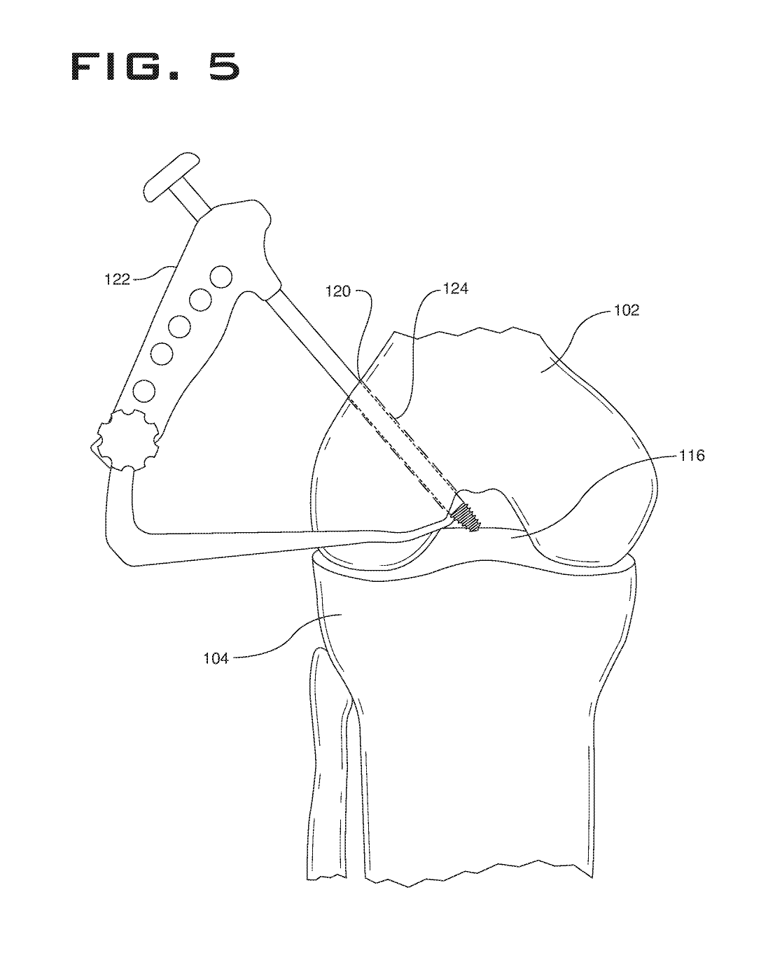

Referring now to FIG. 1, an example of a method of ACL repair with the biologically active suture of the present disclosure is illustrated. In FIG. 1, the femur 102, the tibia 104 and the ACL 108 of a patient's knee capsule 100 are illustrated. For simplicity of illustration, the surrounding tissue and bone (e.g., skin, patella) are omitted from the figure. A first fixation device 106 and a second fixation device 110 are introduced into the knee capsule 100 by means of any technique capable of implanting fixation devices, resulting in the first and second fixation devices 106, 110 being located near or at the insertion sites of the ACL 108. For example, the technique may be an “all-inside” technique as shown in FIG. 1, wherein the first fixation device 106 is located inside a femoral tunnel 124 drilled into the femur 102 and the second fixation device 110 is located inside a tibial tunnel 126 drilled into the tibia 104 from the inside of the joint 116. The technique may alternatively be a trans-tibial technique, as shown in FIG. 2, wherein a drill 122 is used to form a tunnel from the tibial cortex 118 through both of the tibia 104 and femur 102, and out through the femoral cortex 120. In this technique, fixation could be achieved with fixation devices 106, 110 located on the far cortices of the tibia 104 and femur 102, as shown in FIG. 3. Another technique may be a medial port technique, as shown in FIG. 4, wherein a drill 122 is used to form the femoral tunnel 124 from inside the joint 116 through the femur 102 and out through the femoral cortex 120. Finally, the technique may be an “outside-in” technique, as shown in FIG. 5, wherein a drill 122 is used to form the femoral tunnel 124 from the femoral cortex 120 (or tibial cortex 118, not shown) into the joint 116. Advantageously, use of the “outside in” technique may allow the use of smaller tunnels, including tunnels the size of a guide wire, which would disrupt less of the native footprint and subsequent fiber attachments.

Either of the first and second fixation devices 106, 110 may include any devices used to rejoin, re-affix, hold or otherwise partake in the repair of tissue. A non-exhaustive list of such devices includes sutures, surgical arrows, staples, darts, bolts, screws, buttons, anchors, nails, rivets or barbed devices. Either of the first and second fixation devices may be an “all-suture” anchor, in which the anchor construct is formed by the suture itself. The fixation devices 106, 110 may have various shapes, diameters or lengths, and may be comprised of a variety of materials. For example, the fixation devices may be completely, or portions thereof, made from a formulation of poly(lactic-co-glycolic) acid (PLGA), β-Tricalcium phosphate (β-TCP) and calcium sulfate, poly-L-lactic acid-hydroxyapatite (PLLA-HA), poly-D-lactide (PDLA), polyether ether ketone (PEEK) or variants thereof. Biocomposite embodiments of the fixation device made from a combination of PLGA, β-TCP, and calcium sulfate are absorbable by the body, which is beneficial to natural healing. An example formulation of PLGA, β-TCP, and calcium sulfate is described in U.S. Pat. No. 8,545,866, the entirety of which is herein incorporated by reference. Other commonly used material for fixation devices, such as titanium, stainless steel, or combinations thereof, are also contemplated by this disclosure. Fixation devices comprising an angiogenic material, such as those described in U.S. Pat. No. 8,541,027, the entirety of which is herein incorporated by reference, are also contemplated by this disclosure.

As shown in FIGS. 1 and 3, the first fixation device 106 may be attached to the second fixation device 110 by one or more flexible elements, such as a suture 112. The suture 112 is placed so that the suture 112 is passed through the ACL 108 and woven within the tear 128 to repair the wound. The suture 112 may be comprised of a biologically active material. Alternatively, it is contemplated by this disclosure that a biologically active scaffold or scaffold/suture hybrid can be used instead of the suture 112.

The biologically active material of the suture 112 may be comprised of a water soluble or water miscible angiogenic material or precursor thereof in admixture with a non-absorbable hydrophobic polymer. For example, the biologically active material may be an angiogenic material as described in U.S. Pat. No. 8,541,027 and U.S. Publication No. 2010/0040662, the disclosures of which are incorporated by reference herein in their entirety. The angiogenic material so described may advantageously stimulate tissue repair in the area surrounding the ACL tear and release factors that promote angiogenesis. In the case of an all-suture anchor, the suture anchor construct may be comprised of the same biologically-active material as the portion of the suture woven through the tear.

Visible in FIGS. 1, 3 and 6, suture 112 is repeatedly interwoven along a length of the ACL, which may add mechanical stability to the ACL 108 generally. While shown as a uniform somewhat linear plurality of stitching, also contemplated may be stitching patterns along the ACL 108 configured to increase the contact surface area between the ACL 108 and suture 112, and thereby quantity of angiogenic material along the ACL. The pattern may therefore snake further across the ACL 108 and back so as to form a zig-zag or S-shaped pattern along the ACL 108. These may advantageously increase angiogenesis generally throughout the ACL. Stitching may also vary in stitch density, such that the surgeon may increase the stitch density proximate the tear 128 for example. This may further mechanically stabilize the tear 128 as well as focus the angiogenic result proximate the area of particular need. In practice, the inventors envision interlacing this suture 112 first along a length of the ACL with suture passing instruments, before coupling fixation devices to the suture 112.

Fixation devices (106, 110) may include a flexible member portion that does not include an angiogenic material. This flexible member portion may extend along a portion of at least one of the tunnels (124 or 126) and couple to the suture 112 at a location within the tunnel(s). Alternatively suture 112 may include a first portion comprising an angiogenic material configured to extend through the ACL 108 and proximate the tear 128, and a second portion that does not comprise angiogenic material configured to selectively couple to a fixation device. This second portion may not interweave with the ACL 108.

A knot may be tied in the suture 112 at either end of the first and final stitch through the ACL 108 or a clip or cinch used, so as to maintain the suture 112 in place around the tear 128 and within the ACL 108. The knot or clip may keep the suture 112 at a desired tension along the ACL 108, and around the tear 128. Access to the ACL tear 128 and interlacing/knot tying of the suture 112 within the tear 128 may be an arthroscopic procedure, somewhat independent of the bone tunnel formation and may use ports distinct from the bone tunnels 124 and 126.

Visible in FIGS. 2 and 3 is a technique, whereby repair may be achieved with a button style fixation means such as Endobutton®CL (manufactured and sold by Smith & Nephew, Inc., more fully described within U.S. Pat. No. 6,533,802, incorporated herein by reference in its entirety). Shown in FIG. 2, a tunnel in both the tibia and femur is formed using a drill tool, the two tunnels being coaxial. Of note, since the ACL is intended to be preserved, this drill may be offset from the ACL 108, and sufficiently spaced away from the ACL so as minimize further tearing or damage to the ACL 108. This drill may also be smaller in size relative to a drill required during an ACL reconstruction for example. In the case of an ACL reconstruction, a bone hole with a relatively large diameter (φ5 to φ10 mm) is generally required, driven by the size of the tissue graft. Since FIGS. 2 and 3 describe a repair of an in-situ ACL, the inventors envision that a smaller diameter (φ4.5 mm or smaller) may be preferable, sufficient to allow passage to a flexible member only, or a small fixation device. For example, this smaller diameter tunnel may allow a button-style fixation device (with a width of 4 mm) to be passed through the tunnel. Similar to the method described in FIG. 1, that angiogenic suture 112 may be interlaced through a portion of the ACL first, proximate the tear 128, before being coupled to first and second fixations devices, 106 and 110. A knot may also be formed. Devices such as the EndoButton®CL may include a flexible member loop, which may be drawn from a cortical surface through one of the tunnels towards the joint 116 for example, with the button-style device remaining outside the joint 116. Alternatively the angiogenic suture 112 may first be coupled to the fixation device (110 or 106) at a location within the joint 116 and the fixation device moved from the inside of the joint 116 along the bone tunnel to the cortical tissue.

Shown in FIGS. 1-3 the two bone tunnels (124 and 126) are substantially coaxially formed. However, this may by undesirable in some cases, depending on the injury and anatomy of the patient. The “outside-in” method shown in FIG. 5 forms a tunnel by drilling from the outside of the femur 102. Since an important quadriceps exists outside the femur 102, a hole with a large diameter cannot be created; however an ACL repair does not require a bone tunnel sufficiently sized to fit a graft, therefore this “outside-in” method may be particularly advantageous for ACL repair. A surgical method may therefore include drilling a hole from the outside of a femur 102 using a drill bit with a small diameter. Similar to previously described methods, the angiogenic suture 112 may be interlaced through a portion of the ACL proximate the tear 128, independent of the tunnel formation. This interlacing may precede coupling with the first and second fixations devices, 106 and 110. Devices such as the EndoButton®CL may include a flexible member loop, which may be drawn from a cortical surface through one of the tunnels towards the joint 116 for example, with the button-style device remaining outside the joint 116. Alternatively the angiogenic suture 112 may first be coupled to the fixation device (110 or 106) at a location within the joint 116 and the fixation device moved from the inside of the joint 116 along the bone tunnel to the cortical tissue.

The biologically active material may also be at least one of angiogenic material or angiogenic precursor material which is capable of breaking down in vivo to form angiogenic material, wherein the angiogenic material is in admixture with polypropylene. The angiogenic material may comprise one or more of sodium butyrate, butyric acid, butyric acid salt, α-monobutyrin; α-dibutyrin, β-dibutyrin, tributyrin, or hydroxybutyrate, and the butyric acid salt may be one of sodium, potassium, calcium, ammonium, and lithium salts. The angiogenic material may comprise one or more of the following angiogenic factors:

angiogenic peptide growth factors, including autologous, xenogenic, recombinant, and synthetic forms of these, including the vascular endothelial growth factors VEGF 121, 165, 189 and 206; fibroblast growth factors FGF-1, FGF-2, FGF-7 (keratinocyte growth factor); transforming growth factor family (TGF-α, -β, platelet derived growth factors PDGF-AA, PDGF-BB, and PDGF-AB; platelet derived endothelial cell growth factor (PD-ECGF); hypoxia inducible factor-1 (HIF-1); scatter factor (SF, also known as hepatocyte growth factor or HGF); placenta growth factor (PIGF)-1, -2; tumor necrosis factor a (TNF-α); midkine; pleiotrophin; insulin-like growth factor-1; epidermal growth factor (EGF); endothelial cell growth factor (ECGF); endothelial stimulating angiogenic factor (ESAF); connective tissue growth factor (CTGF); CYR61; Angiogenin; or Angiotrophin. The angiogenic material may comprise one or more blood clot breakdown products, including thrombin, heparin, and autologous, allogeneic, xenogeneic, recombinant, and synthetic forms of these materials. The angiogenic material may also comprise one or more of hyaluronan, para-thyroid hormone, angiopoietin 1, del-1, erythropoietin, fas (CD95), follistatin, macrophage migration inhibitory factor, monocyte chemoattractant protein-1, and nicotinamide. The angiogenic precursor material comprises one or more of fibrin, including autologous, allogeneic, xenogeneic, recombinant and synthetic forms thereof, and hyaluronic acid.

The suture 112 may be impregnated (e.g., dipped or soaked) with the biologically active material after manufacture of the suture 112, such that the biologically active material is distributed throughout up to the whole of the suture 112. Alternatively, the biologically active material may be physically incorporated into the main fabric of the suture 112. For example, threads of biologically active material may be braided with polyethylene terephthalate fibers used to produce the suture 112. The biologically active material may be present in an amount that is therapeutically effective for humans.

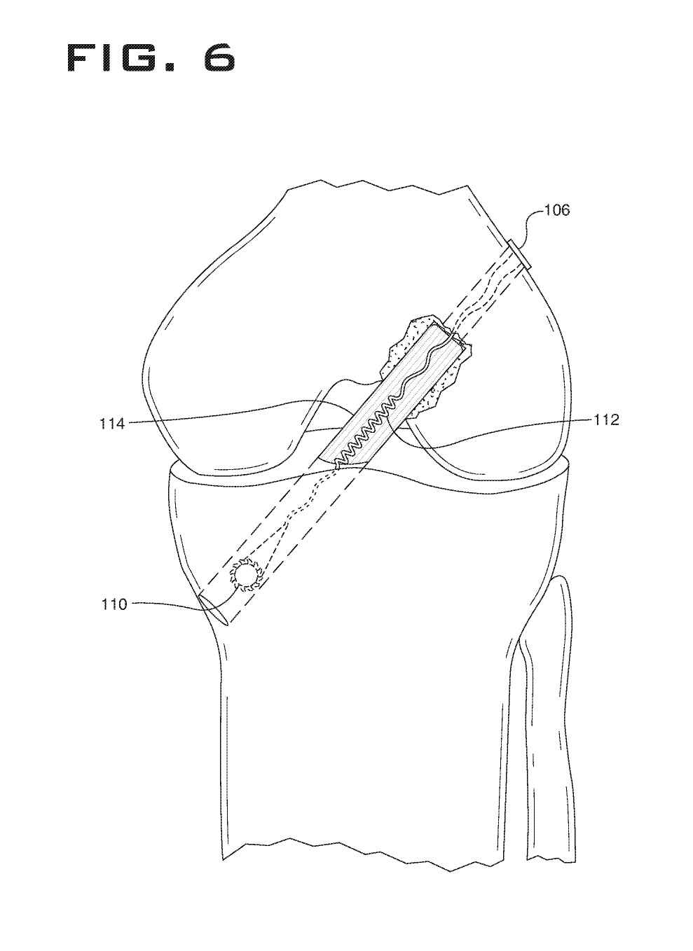

FIG. 6 illustrates the method of ACL repair of this disclosure including a reconstruction using a tissue graft 114, such as a patella tendon, quad tendon, or hamstring. Advantageously, use of the suture 112 material with a biologically active material may help with revascularization/remodeling of the graft 114, and therefore decrease rehabilitation time. Visible in FIG. 6, is a tunnel in both the tibia and femur formed using at least one drill. The tunnels may be coaxial and may be coincident with the ACL insertion site. Of note, since the ACL is intended to be replaced, this drill may be placed so as to be somewhat coincident with the ACL insertion sites, and not offset or spaced away from the ACL as described during ACL repair. Bone tunnels 124 and 126 may have a relatively large diameter (φ5 to φ10 mm) for inserting a tissue graft 114. The diameter (thickness) of the tissue graft 114 may be measured in advance, and bone tunnels with a diameter corresponding (equal) to that size may be created. Furthermore, if a button-style fixation device 106 (with a width of 4 mm) is used, a bone tunnel portion near the femoral cortex having a smaller diameter (φ4.5 mm) may be formed. This allows a flexible element to extend from the tissue graft 114 to the button-style fixation device, while preserving bone tissue. The angiogenic suture 112 may be interlaced through a portion of the tissue graft 114 first, and this may occur external to the knee capsule 100 or joint 116. A knot may be tied at the first and/or last stitch to keep the angiogenic suture 112 in place along the tissue graft 114, as described earlier. In a first example method, the button-style fixation device may be coupled to a first end of the graft, the graft having the angiogenic suture 112 threaded therethrough, and together they may be inserted into the joint 116 via the tibial tunnel and then moved further along and into the femoral tunnel portion from the inside of the joint 116. The button-style fixation device may extend up to subcutaneous tissue via the femoral bone tunnel. At least one flexible member may be fastened to the button-style fixation device configured to couple to a first portion of the tissue graft. A second portion of the tissue graft may be coupled to a second fixation device (106 or 110) within or adjacent the tibial tunnel. This second fixation device may be a screw, configured to couple the tissue graft to walls on the tibial tunnel. The second fixation device may alternatively be a second button-style fixation device. In some example methods, angiogenic suture 112 may couple to fixation devices, whereas in alternative example methods the fixation devices may be coupled solely to ends or portions of the graft and separate from the angiogenic suture 112.

It is contemplated by this disclosure that the methods of ACL repair as described herein may be used as a primary procedure (i.e., employed for the actual repair of the ACL tissue) or may be used in conjunction with other methods of ACL repair, such as ACL reconstruction.

Although the present disclosure has been described with respect to various examples, it would be apparent to one of ordinary skill in the art that various other examples are possible, without departing from the spirit and scope as defined in the appended claims.

What is claimed is:

1. A method of anterior cruciate ligament (ACL) reconstruction, comprising:
passing a suture material through and along a length of a tissue graft, the suture material comprising a water soluble or water miscible, biologically active material or precursor thereof in admixture with a non-absorbable hydrophobic polymer; and
placing the tissue graft and suture material within a first tunnel within a tibia and a second tunnel within a femur;
using a first fixation device, securing a first end of the tissue graft within the first tunnel; and
using a second fixation device, secure a second end of the tissue graft within the second tunnel, wherein the biologically active material stimulates tissue graft remodeling.

2. The method of claim 1, wherein the biologically active material is at least one of angiogenic material or angiogenic precursor material which is capable of breaking down in vivo to form angiogenic material, and wherein the angiogenic material is in admixture with polypropylene.

3. The method of claim 2, wherein the angiogenic material comprises one or more of butyric acid, butyric acid salt, α-monobutyrin; α-dibutyrin, β-dibutyrin, tributyrin, or hydroxybutyrate.

4. The method of claim 3, wherein the butyric acid salt is selected from sodium, potassium, calcium, ammonium, and lithium salts.

5. The method of claim 2, wherein the angiogenic material comprises one or more of the following angiogenic factors: angiogenic peptide growth factors, including autologous, xenogenic, recombinant, and synthetic forms of these, including the vascular endothelial growth factors VEGF 121, 165, 189 and 206; fibroblast growth factors FGF-1, FGF-2, FGF-7 (keratinocyte growth factor); transforming growth factor family (TGF-α, -β, platelet derived growth factors PDGF-AA, PDGF-BB, and PDGF-AB; platelet derived endothelial cell growth factor (PD-ECGF); hypoxia inducible factor-1 (HIF-1); scatter factor (SF, also known as hepatocyte growth factor or HGF); placenta growth factor (PIGF)-1, -2; tumor necrosis factor α (TNF-α); midkine; pleiotrophin; insulin-like growth factor-1; epidermal growth factor (EGF); endothelial cell growth factor (ECGF); endothelial stimulating angiogenic factor (ESAF); connective tissue growth factor (CTGF); CYR61; Angiogenin; or Angiotrophin.

6. The method of claim 2, wherein the angiogenic material comprises one or more blood clot breakdown products, including thrombin, heparin, and autologous, allogeneic, xenogeneic, recombinant, and synthetic forms of these materials.

7. The method of claim 2, wherein the angiogenic material comprises one or more of hyaluronan, para-thyroid hormone, angiopoietin 1, del-1, erythropoietin, fas (CD95), follistatin, macrophage migration inhibitory factor, monocyte chemoattractant protein-1, and nicotinamide.

8. The method of claim 2, wherein the angiogenic precursor material comprises one or more of fibrin, including autologous, allogeneic, xenogeneic, recombinant and synthetic forms thereof, and hyaluronic acid.

9. The method of claim 1, wherein the suture is coated on at least one external surface with the biologically active material.

10. The method of claim 1, wherein the suture has the biologically active material impregnated into at least one region thereof.

11. The method of claim 1, wherein the first fixation device comprises a suture, a surgical arrow, a staple, a dart, a bolt, a screw, a button, an anchor, a nail or a rivet, or a barbed surgical device.

12. The method of claim 1, wherein the second fixation device comprises a suture, a surgical arrow, a staple, a dart, a bolt, a screw, a button, an anchor, a nail or a rivet, or a barbed surgical device.

13. The method of claim 1, wherein the surgical technique is an “all-inside” technique.

14. The method of claim 1, wherein the surgical technique is a trans-tibial technique.

15. The method of claim 1, wherein the surgical technique is a medial port technique.

16. The method of claim 1, wherein the surgical technique is an “outside-in” technique.

17. A method of anterior cruciate ligament (ACL) reconstruction, comprising:
using a surgical technique, drilling a first tunnel within one of a tibia or a femur near a first end of an ACL insertion site;
using the surgical technique, drilling a second tunnel within the other of the tibia or the femur near a second end of the ACL insertion site;
passing a suture material through and along a length of a tissue graft; and
placing the tissue graft and suture material within the first and second tunnels, wherein the suture material is comprised of a water soluble or water miscible, biologically active material or precursor thereof in admixture with a non-absorbable hydrophobic polymer; and
wherein the biologically active material stimulates tissue repair and remodeling.

18. The method of claim 17, wherein the biologically active material is at least one of angiogenic material or angiogenic precursor material which is capable of breaking down in vivo to form angiogenic material, and wherein the angiogenic material is in admixture with polypropylene.

19. The method of claim 17.wherein the angiogenic material comprises one or more of butyric acid, butyric acid salt, α-monobutyrin; α-dibutyrin, β-dibutyrin, tributyrin, or hydroxybutyrate.

20. A kit for ligament reconstruction comprising:
a drill for preparing a first tunnel within a femur;
a first fixation device;
a suture material comprising an angiogenic material to stimulate tissue graft repair and remodeling, wherein the suture material is configured to be passed through and along a length of a tissue graft and wherein the first fixation is configured to secure the tissue graft within the first tunnel.
